(19) United States
(12) Patent Application Publication
(10) Pub. No.: US 2026/0108540 A1
(43) Pub. Date: Apr. 23, 2026

(54) NOVEL LIQUID ORAL FORMULATIONS OF CANNABIDIOL

(71) Applicant: LEIUTIS PHARMACEUTICALS LLP, Hyderabad (IN)

(72) Inventors: Chandrashekhar KOCHERLAKOTA, Secunderabad (IN); Srinivas ARUTLA, Hyderabad (IN)

(21) Appl. No.: 19/120,456

(22) PCT Filed: Jun. 24, 2023
(86) PCT No.: PCT/IB2023/056536

§ 371 Date: Apr. 11, 2025

(30) Foreign Application Priority Data
Oct. 12, 2022 (IN) 202241058354

(51) Int. Cl. A61K 31/00; A61K 9/00; A61K 9/08; A61K 47/10; A61K 47/14; A61K 47/22; A61K 47/26; A61K 47/44

(52) U.S. Cl. CPC A61K 31/658; A61K 9/0053; A61K 9/08; A61K 47/10; A61K 47/14; A61K 47/22; A61K 47/26; A61K 47/44

(57) ABSTRACT

The present invention relates to stable liquid oral pharmaceutical formulations of cannabidiol (CBD) and also discloses methods of producing stabilized formulations of cannabidiol using cannabidiol from a synthetic source. The stable formulations of the present invention comprise of synthetic CBD and one or more pharmaceutically acceptable excipients selected from solvents, oils, organoleptic modifiers, buffers, surfactants etc.

### FIELD OF THE INVENTION

The present invention relates to stable liquid oral pharmaceutical formulations of cannabidiol (CBD). The invention further discloses methods of producing a stabilized formulation of cannabidiol using cannabidiol from a synthetic source.

### BACKGROUND OF THE INVENTION

Natural cannabis for medicinal use is not just a single entity but encompasses a diversity of products. It contains approximately 500 molecules, including approximately 100 plant-derived cannabis compounds (phytocannabinoids), terpenes and flavonoids. The best characterised phytocannabinoids are Δ9-tetrahydrocannabinol (THC) and cannabidiol (CBD). The intoxicating effect of cannabis is due to THC whereas CBD does not have any such effect.

Cannabidiol can be obtained from natural origin or produced synthetically. There are many disadvantages associated to use of CBD from natural origin. One major disadvantage is that the CBD is co-extracted with many other components. Further, the qualitative and quantitative composition of natural CBD depends on the origin, the species of plant and purification procedures. This often results in products with varying characteristics.

Additionally, the process for purification is often complex and time consuming. The undesirable constituents are removed from natural CBD by a treatment known as “winterization.” This is a time consuming and expensive process wherein the extract is placed in a freezer at −20° to −80° C. for 24-48 h causing components with a higher melting point such as waxes, triglycerides, and chlorophyll to precipitate.

Even after such expensive purification processes, the natural CBD contains many major impurities like Cannabidivarin (CBDV), Tetrahydrocannabinol (THC), butyl analog of CBD (CBD-C4), Cannabidiolic acid (CBDA), terpenes etc.

Synthetic CBD, on the other hand, has consistent quality attributes and can be manufactured in controlled conditions. It is also an economical alternative to natural CBD. But synthetic CBD is also prone to degradation, particularly when exposed to air, light and high temperatures.

FDA approved CBD is available as Epidiolex®, an oral solution (100 mg/mL) for treating seizures associated with Lennox-Gastaut syndrome, Dravet syndrome, or tuberous sclerosis complex. The CBD used in this product is extracted from hemp inflorescence.

U.S. Pat. No. 9,474,726 (B2) to GW Pharma Ltd et al., discloses a method of treating treatment-resistant epilepsy using CBD in combination with one or more anti-epileptic drugs (AEDs). The possible impurities detected were: CBDA (NMT 0.15% w/w), CBDV (NMT 1.0% w/w), CBD-C4 (NMT 0.5% w/w) and THC (NMT 0.15% w/w).

U.S. Pat. No. 10,709,673 to GW Pharma Ltd et al., discloses the use of a highly purified cannabis extract comprising at least about 95% (w/w) cannabidiol (CBD), less than 0.15% THC and up to 1% CBDV.

U.S. patent application No. 20220202738 to GW Pharma Ltd et al., discloses method of treating absence seizures using CBD of specified purity.

U.S. Pat. No. 11,331,279 to Kiran Kumar et al., and PCT publication no. WO2021046628 (A1) to Blagoja et al., discloses stable oral cannabinoid formulations of synthetic CBD, comprising antioxidants. This patent teaches that stable formulations are possible only using antioxidants.

### SUMMARY OF THE INVENTION

There is nothing in the art to suggest a method of making a formulation with synthetic CBD or a method of stabilizing such formulation without use of antioxidants.

One aspect of the present invention is to provide a liquid oral formulations of CBD, wherein the CBD is obtained from a synthetic source.

Another aspect of the present invention is to provide a liquid oral formulation of CBD, wherein the CBD is from a synthetic source and the formulation is completely free of THC.

Yet another aspect of the present invention is to provide a liquid oral formulation of CBD, wherein the CBD is obtained from a synthetic source and the formulation is completely free of THC and CBDV.

Yet another aspect of the present invention is to provide a solution formulation of CBD, wherein the CBD is obtained from a synthetic source and the formulation is completely free of THC, CBDV and terpenes.

Another aspect of the present invention is to provide a solution formulation of CBD wherein CBD is obtained from a synthetic source and the formulation is completely free of antioxidants.

Yet another aspect of the invention is to provide a solution formulation of CBD, wherein the CBD is obtained from a synthetic source and the formulation is free of THC and CBDV and antioxidants.

### DETAILED DESCRIPTION OF THE INVENTION

CBD in the present invention relates to synthetic cannabidiol. Cannabidiol is present in the range from 2%-60% w/v of the total formulation and preferably from 5%-20% w/v. The chemical name of CBD is 2-[(1R,6R)-3-Methyl-6-(1-methylethenyl)-2-cyclohexen-1-yl]-5-pentyl-1,3-benzenediol and has the following structure:

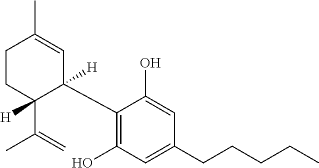 [SMILES: C=C(C)[C@@H]1CCC(C)=C[C@H]1c1c(O)cc(CCCCC)cc1O]

The term “completely free” in the present invention relates to formulations with no or zero amount or less than the quantifiable limit of the specified impurities for e.g. delta 9-tetrahydrocannabinol (THC) and CBDV.

The term “substantially free” in the present invention relates to values less than 0.3%. For example, the term substantially free of impurity A means impurity A is less than 0.3%.

Natural CBD is a mixture of several different ingredients. Apart from THC and CBDV, it may also contain several other coexisting compounds. These compounds help in stabilising the natural CBD but are associated with their own undesirable pharmacological effects. Some of the impurities reported in cannabidiol preparations are:
Impurity A: Cannabidiolic Acid (CBDA)
Chemical Name: 2,4-Dihydroxy-3-[(1R,6R)-6-isopropenyl-3-methyl-2-cyclohexen-1-yl]-6-pentylbenzoic acid
Structure:

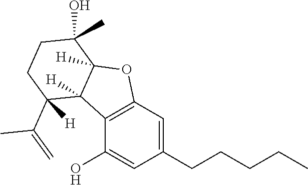

### Impurity B: Cannabidibutol, Cannabidiol-C4, CBD-C4, CBDB

Chemical Name: (1R-trans)-5-butyl-2-[3-methyl-6-(1-methylethenyl)-2-cyclohexen-1-yl]-1,3-benzenediol
Structure:

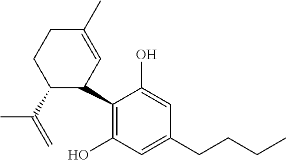

Impurity C: Cannabielsoin (CBE)
Chemical Name: (5aS,6S,9R,9aR)-9-Isopropenyl-6-methyl-3-pentyl-5a,6,7,8,9,9a-hexahydrodibenzo[b,d]furan-1,6-diol
Structure:

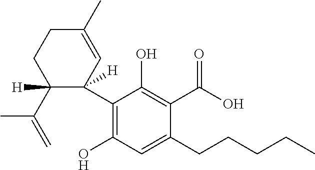 [SMILES: C=C(C)[C@@H]1CCC(C)=C[C@H]1c1c(O)cc(CCCCC)c(C(=O)O)c1O]

Impurity D: Cannabichromene (CBC)
Chemical Name: 2-Methyl-2-(4-methyl-3-penten-1-yl)-7-pentyl-2H-chromen-5-ol
Structure:

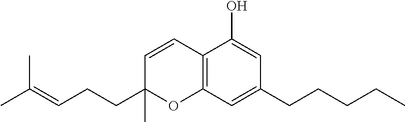 [SMILES: CCCCCc1cc(O)c2c(c1)OC(C)(CCC=C(C)C)C=C2]

Impurity E: Cannabigerol (CBG)
Chemical Name: 2-[(2E)-3,7-Dimethyl-2,6-octadien-1-yl]-5-pentyl-1,3-benzenediol
Structure:

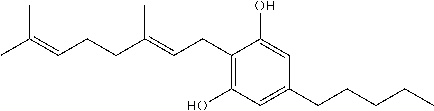 [SMILES: CCCCCc1cc(O)c(C/C=C(\C)CCC=C(C)C)c(O)c1]

Impurity F: Cannabinol (CBN)
Chemical Name: 6,6,9-Trimethyl-3-pentyl-6H-benzo[c]chromen-1-ol
Structure:

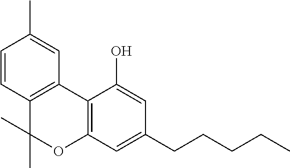 [SMILES: CCCCCc1cc(O)c2c(c1)OC(C)(C)c1ccc(C)cc1-2]

Impurity G: 7-hydroxycannabidiol (OH CBD)
Chemical Name: 2-[(1R,6R)-3-(Hydroxymethyl)-6-isopropenyl-2-cyclohexen-1-yl]-5-pentyl-1,3-benzenediol
Structure:

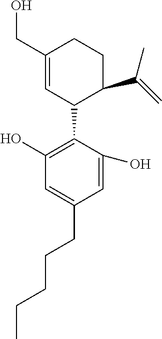

Impurity H: Tetrahydrocannabidavarin (THCV)
Chemical Name: 6,6,9-Trimethyl-3-propyl-6a,7,8,10a-tetrahydro-6H-benzo[c]chromen-1-ol
Structure:

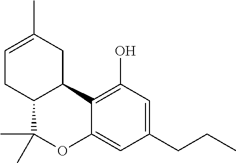 [SMILES: CCCc1cc(O)c2c(c1)OC(C)(C)[C@@H]1CC=C(C)C[C@@H]21]

Impurity I: δ9-Tetrahydrocannabinolic acid (THCA)
Chemical name: (6aR, 10aR)-1-Hydroxy-6,6,9-trimethyl-3-pentyl-6a,7,8,10a-tetrahydro-6H-benzo[c]chromene-2-carboxylic acid
Structure:

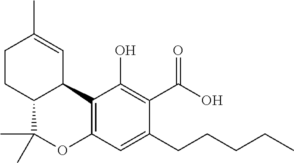 [SMILES: CCCCCc1cc2c(c(O)c1C(=O)O)[C@@H]1C=C(C)CC[C@H]1C(C)(C)O2]

Impurity J: Cannabinol (CBN)
Chemical name: 6,6,9-Trimethyl-3-pentyl-6H-benzo[c]chromen-1-ol
Structure:

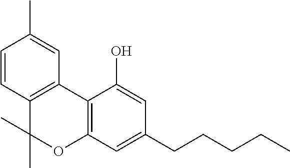 [SMILES: CCCCCc1cc(O)c2c(c1)OC(C)(C)c1ccc(C)cc1-2]

The commercial formulation of CBD in the US uses CBD from a natural source and has a content of THC less than 0.1% and CBDV of about 0.5% and total impurities of about 1.5%. The actual specification may be much higher, for example, CBDV of about 1%.

Unlike the formulations known in the prior art, the cannabidiol formulations of the present invention are substantially free of impurities listed above, namely, impurity A, impurity B, impurity C, impurity D, impurity E, impurity F, impurity G, impurity H, impurity I, impurity J. More particularly, the present formulations are completely free of CBDV and THC. The total impurities in the formulation of the present invention are less than 0.5%.

THC and CBDV have the following structure:
Cannabidivarin (CBDV)
Chemical name: 2-[(1R,6R)-6-Isopropenyl-3-methyl-2-cyclohexen-1-yl]-5-propyl-1,3-benzenediol
Structure:

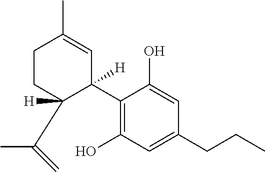 [SMILES: C=C(C)[C@@H]1CCC(C)=C[C@H]1c1c(O)cc(CCC)cc1O]

Tetrahydrocannabinol (THC)
Chemical name: 6aR, 10aR)-delta-9-Tetrahydrocannabinol; (−)-trans-Δ9-Tetrahydrocannabinol
Structure:

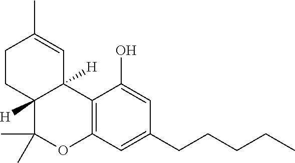 [SMILES: CCCCCc1cc(O)c2c(c1)OC(C)(C)[C@@H]1CCC(C)=C[C@@H]21]

The impurities were analysed using a standard analytical technique as disclosed herein.

Formulation for oral administration in the context of the present invention means a formulation that is administered orally such as a solution or suspension in an aqueous or non-aqueous liquid forms, an oil-in-water or water-in-oil liquid emulsion, an elixir or syrup, linctus, drops, tincture or as a slurry or a spray.

The present inventors were successful in formulating oral CBD compositions using synthetic CBD that are completely free of THC and CBDV. These formulations are substantially free of any of the previously listed impurities as described namely, impurity A, impurity B, impurity C, impurity D, impurity E, impurity F, impurity G, impurity H, impurity I, impurity J.

Unlike the formulation of prior art which are stabilised only by using antioxidants, surprisingly these formulations were found to be stable even in the absence of antioxidants.

The present invention may comprise of solvents and/or co-solvents, oils, organoleptic modifiers, surfactants, preservatives, chelating agents and buffers.

The oil(s) in the present invention may range up to 90% w/v and are selected form soybean oil, MCT oil, sesame oil, olive oil, corn oil, vitamin E, grapeseed oil, walnut oil, avocado oil, flax seed oil, coconut oil, olive oil, hemp seed oil, ginger oil.

The solvent(s) or co solvent(s) in the present invention may range up to 90% w/v and are selected form but not limited to ethanol, N-methyl-2-pyrrolidone (NMP), tertiary butyl alcohol (TBA), glycerol, propylene glycol, polyethylene glycol and combinations thereof.

The surfactant(s) in the present invention are selected form but not limited to amphiphilic surfactants, lipophilic surfactants. They can be selected from polysorbates, Span-20, Span-60 and Span-80, cremophor EL, anionic bile salts sodium cholate, sodium glycocholate, sodium tauro cholate, sodium tauro deoxy cholate, sodium lauryl sulphate, docusate sodium etc.

The chelating agents(s) in the present invention are selected form but not limited to like 1,4,7,10-tetraazacyclododecane-1,4,7,10-tetraacetic acid (DOTA), disodium EDTA and preservatives can be selected from methyl paraben, propyl paraben, chlorobutanol.

The buffer(s) in the present invention are selected form but not limited L-histidine, L-arginine, tris base, meglumine, glycine, sodium succinate, diethanol amine, aspartic acid, glutamic acid, alanine, sodium acetate, sodium ascorbate, sodium citrate, citric acid, succinic acid, triethanolamine, boric acid, sodium hydroxide, sodium bicarbonate etc.

The present invention may further comprise flavouring agents, colouring agents and sweeteners. When sweetener(s) are used, they are typically present in the range from 0-10% w/v.

The formulation of present invention is completely free of antioxidants.

When provided as in above-mentioned dosage forms the formulation may be dispensed in bottles optionally with syringes, in sachets, ampoules, vials, droppers, prefilled syringes made of glass, rubber, or plastic such as polyvinyl chloride, polypropylene, polystyrene and polyethylene and also metals such as steel, aluminium and various alloys. These containers are further sealed, closed or packed using stoppers, closures, lids, seals, and caps made of various materials.

A preferred embodiment of the present invention may be in the form of an oral liquid formulation comprising
(i) synthetic cannabidiol,
(ii) one or more solvents selected from the group comprising ethanol, N-methyl-2-pyrrolidone (NMP), tertiary butyl alcohol (TBA), glycerol, propylene glycol, polyethylene glycol,
wherein the formulation is
   (a) completely free of antioxidants,
   (b) completely free of THC,
   (c) completely free of CBDV.

Another embodiment of the present invention may be in the form of an oral liquid formulation comprising
(i) synthetic cannabidiol,
(ii) one or more solvents selected from the group comprising ethanol, N-methyl-2-pyrrolidone (NMP), tertiary butyl alcohol (TBA), glycerol, propylene glycol, polyethylene glycol,
wherein the formulation is
   (a) completely free of antioxidants,
   (b) completely free of THC,
   (c) completely free of CBDV,
   (d) substantially free of impurities such as impurity A, impurity B, impurity C, impurity D, impurity E, impurity F, impurity G, impurity H, impurity I, and impurity J.

The present invention formulation can be used to treat epilepsy, anxiety, depression, inflammation and pain. Compared to solid oral formulations, the invention formulations have the advantage that they are in liquid form and hence can be easily administered.

The following are examples intended to illustrate the present invention but are not considered to limit the scope of the present invention.

### Example 1

| S. No | Ingredients | Qty (mg/mL) | Qty (% w/v) |
|---|---|---|---|
| 1 | cannabidiol | 100.00 | 10 |
| 2 | sesame oil | 736.00 | 73.6 |
| 3 | ethanol absolute | 79.00 | 7.9 |
| 4 | sucralose | 0.70 | 0.070 |
| 5 | strawberry flavour | 0.27 | 0.027 |

### Manufacturing Process

1. Weighed quantity of sesame oil was taken in a manufacturing vessel.
2. Into a separate steel container attached with a propeller mixer, ethanol absolute was added followed by addition of sucralose with continuous mixing.
3. While stirring, ethanol absolute and sucralose mixture was added to the above manufacturing vessel, followed by addition of cannabidiol and with continuous mixing.
4. Strawberry flavour was added to the above manufacturing vessel and stirred continuously for 5 minutes.
5. The above solution was collected, filtered through 50 microns filter and required quantity was filled into bottles followed by capping and sealing the filled bottles using PP28 mm Medi-Loc Tamper Evident Two-Piece CRC Closure.

Analytical procedure: The formulation of the present invention was analyzed using a specific HPLC standardized method using column: Zobrax Eclipse XDB C8, (250×4.6 mm), 5 μm or equivalent and solvent system comprising acetonitrile: water, for an elution time of 70 minutes.

TABLE 1
Stability data of example 1 in triplicate at temperature condition of 25 ± 2° C./75 ± 5% RH.

| | Formulations | | | | | | | | | | | |
|---|---|---|---|---|---|---|---|---|---|---|---|---|
| | 21A | | 21B | | 22A | | 22B | | 23A | | 23B | |
| | Time point | | | | | | | | | | | |
| | I₀ | 3 M | I₀ | 3 M | I₀ | 3 M | I₀ | 3 M | I₀ | 3 M | I₀ | 3 M |
| Assay % | 98.6 | 97.7 | 97.2 | 97.1 | 97.7 | 97.5 | 96.5 | 99.4 | 95.7 | 97 | 95.6 | 99.4 |

Related Substances (Impurities)

| THC | Less than the limit of Quantitation | | | | | | | | | | | |
|---|---|---|---|---|---|---|---|---|---|---|---|---|
| CBDV | Less than the limit of Quantitation | | | | | | | | | | | |
| Unspecified | 0.03 | 0.05 | 0.07 | 0.06 | 0.03 | 0.08 | 0.03 | 0.07 | 0.03 | 0.07 | 0.03 | 0.06 |
| impurities | | | | | | | | | | | | |
| Total | 0.1 | 0.3 | 0.2 | 0.3 | 0.1 | 0.3 | 0.1 | 0.3 | 0.1 | 0.3 | 0.1 | 0.3 |
| Impurities | | | | | | | | | | | | |

Note:
I₀ indicates initial values
* indicates unspecified impurities - impurity A, impurity B, impurity C, impurity D, impurity E, impurity F, impurity G, impurity H, impurity I, and impurity J.

TABLE 2
Stability data of example 1 in triplicate at temperature condition of 30 ± 2° C./75 ± 5% RH.

| | Formulations | | | | | | | | | | | |
|---|---|---|---|---|---|---|---|---|---|---|---|---|
| | 21A | | 21B | | 22A | | 22B | | 23A | | 23B | |
| | Time point | | | | | | | | | | | |
| | I₀ | 3 M | I₀ | 3 M | I₀ | 3 M | I₀ | 3 M | I₀ | 3 M | I₀ | 3 M |
| Assay % | 98.6 | 97.7 | 97.2 | 95.9 | 97.7 | 95.9 | 96.5 | 96 | 95.7 | 96.3 | 95.6 | 95.9 |

Related Substances (Impurities)

| THC | Less than the limit of Quantitation | | | | | | | | | | | |
|---|---|---|---|---|---|---|---|---|---|---|---|---|
| CBDV | Less than the limit of Quantitation | | | | | | | | | | | |
| Unspecified | 0.03 | 0.07 | 0.07 | 0.05 | 0.03 | 0.07 | 0.03 | 0.05 | 0.03 | 0.05 | 0.03 | 0.07 |
| impurities* | | | | | | | | | | | | |
| Total | 0.1 | 0.3 | 0.2 | 0.3 | 0.1 | 0.3 | 0.1 | 0.3 | 0.1 | 0.3 | 0.1 | 0.3 |
| Impurities | | | | | | | | | | | | |

Note:
I₀ indicates initial values
*indicates unspecified impurities -impurity A, impurity B, impurity C, impurity D, impurity E, impurity F, impurity G, impurity H, impurity I, and impurity J.

TABLE 3
Stability data of example 1 in triplicate at temperature condition of 40 ± 2° C./75 ± 5% RH.

| | Formulations | | | | | | | | | | | |
|---|---|---|---|---|---|---|---|---|---|---|---|---|
| | 21A | | 21B | | 22A | | 22B | | 23A | | 23B | |
| | Time point | | | | | | | | | | | |
| | I₀ | 3 M | I₀ | 3 M | I₀ | 3 M | I₀ | 3 M | I₀ | 3 M | I₀ | 3 M |
| Assay % | 98.6 | 97.3 | 97.2 | 96.7 | 97.7 | 97 | 96.5 | 97.1 | 95.7 | 96.5 | 95.6 | 97 |

Related Substances (Impurities)

| THC | Less than the limit of Quantitation | | | | | | | | | | | |
|---|---|---|---|---|---|---|---|---|---|---|---|---|
| CBDV | Less than the limit of Quantitation | | | | | | | | | | | |
| Unspecified | 0.03 | 0.07 | 0.07 | 0.06 | 0.03 | 0.07 | 0.03 | 0.06 | 0.03 | 0.07 | 0.03 | 0.06 |
| impurities | | | | | | | | | | | | |
| Total | 0.1 | 0.3 | 0.2 | 0.3 | 0.1 | 0.3 | 0.1 | 0.2 | 0.1 | 0.3 | 0.1 | 0.2 |
| Impurities | | | | | | | | | | | | |

Note:
I₀ indicates initial values
* indicates unspecified impurities - impurity A, impurity B, impurity C, impurity D, impurity E, impurity F, impurity G, impurity H, impurity I, and impurity J.

### Example 2

| S. No | Ingredients | Qty (mg/mL) | Qty (% w/v) |
|---|---|---|---|
| 1 | cannabidiol | 100 | 10.00 |
| 2 | MCT oil | 650 | 65.00 |
| 3 | sucralose | 0.70 | 0.070 |
| 4 | strawberry flavour | 0.27 | 0.027 |
| 5 | ethanol absolute | Q.S to 1 ml | Q.S to 100% |

### Manufacturing Process

1. Weighed quantity of MCT oil was taken in a manufacturing vessel.
2. Into a separate steel container attached with a propeller mixer, required quantity of ethanol absolute was added followed by addition of sucralose with continuous mixing.
3. While stirring, ethanol absolute and sucralose mixture was added to the above manufacturing vessel, followed by addition of cannabidiol and with continuous mixing.
4. Strawberry flavour was added to the above manufacturing vessel and stirred continuously for 5 minutes, further final volume was adjusted using ethanol.
5. The above solution was collected, filtered through 50 microns filter and required quantity was filled into bottles followed by capping and sealing the filled bottles using PP28 mm Medi-Loc Tamper Evident Two-Piece CRC Closure.

### Example 3

| S. No | Ingredients | Qty (mg/mL) | Qty (% w/v) |
|---|---|---|---|
| 1 | cannabidiol | 100 | 10 |
| 2 | cremophor | 300 | 30 |
| 3 | sucralose | 0.80 | 0.080 |
| 4 | vanilla flavour | 0.27 | 0.027 |
| 5 | ethanol absolute | Q.S to 1 ml | Q.S to 100% |

### Manufacturing Process

1. Weighed quantity of cremophor was taken in a manufacturing vessel.
2. Into a separate steel container attached with a propeller mixer, ethanol absolute was added followed by addition of sucralose with continuous mixing.
3. While stirring required quantity of ethanol absolute and weighed quantity of sucralose mixture was added to the above manufacturing vessel, followed by addition of cannabidiol and with continuous mixing.
4. Vanilla flavour was added to the above manufacturing vessel and stirred continuously for 5 minutes, further final volume was adjusted using ethanol.
5. The above solution was collected, filtered through 50 microns filter and required quantity was filled into bottles followed by capping and sealing the filled bottles using PP28 mm Medi-Loc Tamper Evident Two-Piece CRC Closure.

### Example 4

| S. No | Ingredients | Qty (mg/mL) | Qty (% w/v) |
|---|---|---|---|
| 1 | cannabidiol | 100 | 10 |
| 2 | N-Methyl- | 100 | 10 |
| | Pyrrolidone | | |
| 3 | sucralose | 0.80 | 0.080 |
| 4 | vanilla flavour | 0.27 | 0.027 |
| 5 | ethanol absolute | Q.S to 1 ml | Q.S to 100% |

### Manufacturing Process

1. Weighed quantity of N-Methyl-Pyrrolidone was taken in a manufacturing vessel.
2. Into a separate steel container attached with a propeller mixer, ethanol absolute was added followed by addition of sucralose with continuous mixing.
3. While stirring, ethanol absolute and sucralose mixture was added to the above manufacturing vessel, followed by addition of cannabidiol and with continuous mixing.
4. Vanilla flavour was added to the above manufacturing vessel and stirred continuously for 5 minutes, further final volume was adjusted using ethanol.
5. The above solution was collected, filtered through 50 microns filter and required quantity was filled into bottles followed by capping and sealing the filled bottles using PP28 mm Medi-Loc Tamper Evident Two-Piece CRC Closure.

### Example 5

| S. No | Ingredients | Qty (mg/mL) | Qty (% w/v) |
|---|---|---|---|
| 1 | cannabidiol | 100 | 10 |
| 2 | N-methyl pyrrolidone | 30 | 3 |
| 3 | propylene glycol | 100 | 10 |
| 4 | sucralose | 0.80 | 0.080 |
| 5 | vanilla flavour | 0.27 | 0.027 |
| 6 | ethanol absolute | Q.S to 1 ml | Q.S to 100% |

### Manufacturing Process

1. Weighed quantity of N-Methyl-pyrrolidone was taken in a manufacturing vessel.
2. Into a separate steel container attached with a propeller mixer, required quantity of ethanol absolute was added followed by addition of sucralose with continuous mixing.
3. While stirring, ethanol absolute and sucralose mixture was added to the above manufacturing vessel along with weighed quantity of propylene glycol, followed by addition of cannabidiol and with continuous mixing.
4. Vanilla flavour was added to the above manufacturing vessel and stirred continuously for 5 minutes, further final volume was adjusted using ethanol.
5. The above solution was collected, filtered through 50 microns filter and required quantity was filled into bottles followed by capping and sealing the filled bottles using PP28 mm Medi-Loc Tamper Evident Two-Piece CRC Closure.

We claim:

1. A liquid oral formulation of cannabidiol comprising:
(i) synthetic cannabidiol,
(ii) one or more solvents selected from the group comprising ethanol, N-methyl-2-pyrrolidone (NMP), tertiary butyl alcohol (TBA), glycerol, propylene glycol, polyethylene glycol, wherein the formulation is
(a) completely free of antioxidants,
(b) completely free of THC,
(c) completely free of CBDV.

2. The formulation of claim 1, wherein the formulation is substantially free of impurities A, B, C, D, E, F, G, H, I and J.

3. The formulation of claim 1, wherein the formulation is a solution.

4. The formulation of claim 1, wherein the formulation is a suspension.

5. The formulation of claim 1, wherein the formulation is an oral spray.

6. The formulation of claim 1, wherein the formulation is an emulsion.

7. The formulation of claim 1, wherein the formulation additionally comprises a buffer.

8. The formulation of claim 1, additionally comprising an oil selected from the group of soybean oil, olive oil, corn oil, vitamin E, grapeseed oil, walnut oil, avocado oil, flax seed oil, coconut oil, olive oil, hemp seed oil, ginger oil.

9. The formulation of claim 1, wherein the proportion of cannabidiol is 2% to 50% w/v.

10. A liquid oral formulation of cannabidiol comprising:
(i) synthetic cannabidiol,
(ii) one or more solvents selected from the group comprising ethanol, N-methyl-2-pyrrolidone (NMP), tertiary butyl alcohol (TBA), glycerol, propylene glycol, polyethylene glycol,
(iii) an oil selected from the group comprising soyabean oil, olive oil, corn oil, vitamin E, grapeseed oil, walnut oil, avocado oil, flax seed oil, coconut oil, olive oil, hemp seed oil, ginger oil,
wherein the formulation is
(a) completely free of antioxidants,
(b) completely free of THC,
(c) completely free of CBDV.

11. An oral spray formulation of cannabidiol comprising:
(i) synthetic cannabidiol,
(ii) one or more solvents selected from the group comprising ethanol, N-methyl-2-pyrrolidone (NMP), tertiary butyl alcohol (TBA), glycerol, propylene glycol, polyethylene glycol, wherein the formulation is
(a) completely free of antioxidants,
(b) completely free of THC,
(c) completely free of CBDV, and
wherein the formulation is packed in a suitable container for effective delivery of spray.

12. An oral spray formulation of cannabidiol comprising:
(i) synthetic cannabidiol
(ii) one or more solvents selected from the group comprising ethanol, N-methyl-2-pyrrolidone (NMP), tertiary butyl alcohol (TBA), glycerol, propylene glycol, polyethylene glycol,
(iii) an oil selected from the group comprising soyabean oil, olive oil, corn oil, vitamin E, grapeseed oil, walnut oil, avocado oil, flax seed oil, coconut oil, olive oil, hemp seed oil, ginger oil,
wherein the formulation is
(a) completely free of antioxidants,
(b) completely free of THC
(c) completely free of CBDV, and
wherein the formulation is packed in a suitable container for effective delivery of spray.

13. An oral solution formulation of cannabidiol comprising:
(i) synthetic cannabidiol,
(ii) one or more solvents selected from the group comprising ethanol, N-methyl-2-pyrrolidone (NMP), tertiary butyl alcohol (TBA), glycerol, propylene glycol, polyethylene glycol, wherein the formulation is
(a) completely free of antioxidants,
(b) completely free of THC,
(c) completely free of CBDV, and
(d) substantially free of impurities A, B, C, D, E, F, G, H, I and J.
